(19) United States
(12) Patent Application Publication
(10) Pub. No.: US 2005/0245948 A1
(43) Pub. Date: Nov. 3, 2005

(54) Corneal marker

(76) Inventor: Steve Khalaj, Laguna Hills, CA (US)

Correspondence Address: IRELL & MANELLA LLP, 840 NEWPORT CENTER DRIVE, SUITE 400, NEWPORT BEACH, CA, 92660, US

(21) Appl. No.: 10/834,612

(22) Filed: Apr. 28, 2004

(51) Int. Cl. A61F 9/00

(52) U.S. Cl. 606/166

(57) ABSTRACT

A corneal marker for marking a cornea. The marker may create markings on the cornea that locate where an electrode tip is to be inserted to perform an ophthalmic procedure. The corneal marker includes a plurality of markers coupled to a frame. An actuator may be depressed to move-the markers into contact with the cornea. By way of example, the markers may be arranged in circular patterns 6, 7 and 8 millimeters about a center of the cornea. The corneal marker can simultaneously create markings in 6, 7 and 8 millimeter circles by merely depressing the actuator.

### BACKGROUND OF THE INVENTION

1. Field of the Invention

The present invention relates to a corneal marker used to mark a cornea during an ophthalmic medical procedure.

2. Background

Refractec, Inc. of Irvine California, the assignee of the present application, has developed a system to correct hyperopia and presbyopia with a thermokeratoplasty probe that is connected to a console. The probe has a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The circular pattern of denatured areas will tighten the stroma and decrease the radius of curvature of the cornea. The procedure is taught by Refractec under the service marks CONDUCTIVE KERATOPLASTY and CK.

The surgeon typically uses a corneal marker to create an ink ring on the cornea to mark the location where the electrode tip is to be inserted. The corneal marker can be centered with a light ring that is projected onto the cornea. The denatured areas are typically created in circular rings 6, 7 and 8 millimeters about the cornea. Each ring of denatured areas requires a separate corneal marker. The surgeon needs a corneal marker to create an ink ring at 6 millimeters, a different corneal marker to create an ink ring at 7 millimeters, and yet another corneal marker to mark a ring at 8 millimeters. Each corneal marker must be centered and then applied to the cornea. Using individual markers increases both the time required to perform a CK procedure, and the possibility of misalignment of the marker.

### BRIEF SUMMARY OF THE INVENTION

A corneal marker that includes a plurality of markers coupled to a frame. The marker may also have an actuator coupled to the frame and the markers.

### BRIEF DESCRIPTION OF THE DRAWINGS

FIG. 1 is a cross-sectional view showing a corneal marker on a cornea;

FIG. 2 is a perspective view of a thermokeratoplasty system;

FIG. 3 is a graph showing a waveform that is provided by a console of the system;

FIG. 4 is an enlarged view of a tip inserted into a cornea;

FIG. 5 is a top view showing a pattern of denatured areas of the cornea;

FIG. 6 is a perspective view of a handle used to align and actuate the corneal marker;

FIG. 7 is a perspective view showing the handle being used to align the corneal marker onto a cornea;

FIG. 8 is a perspective view showing the handle being used to actuate the corneal marker.

### DETAILED DESCRIPTION

Disclosed is a corneal marker for marking a cornea. The marker may create markings on the cornea that locate where an electrode tip is to be inserted to perform an ophthalmic procedure. The corneal marker includes a plurality of markers coupled to a frame. An actuator may be depressed to move the markers into contact with the cornea. By way of example, the markers may be arranged in circular patterns 6, 7 and 8 millimeters about a center of the cornea. The corneal marker can simultaneously create markings in 6, 7 and 8 millimeter circles by merely depressing the actuator.

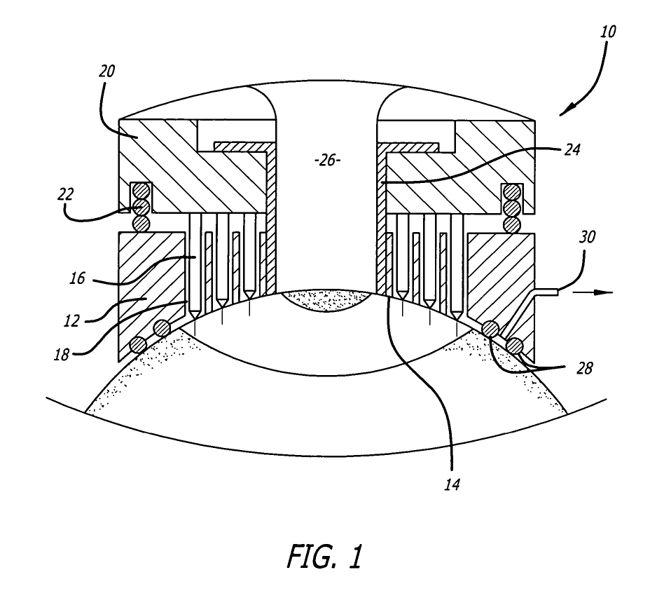

Referring to the drawings more particularly by reference numbers, FIG. 1 shows a corneal marker 10. The corneal marker 10 creates markings on a cornea. The marker 10 may include a frame 12 with a bottom surface 14 that conforms to the shape of the cornea. A plurality of markers 16 may extend through openings 18 in the frame 12. By way of example, the openings 18 may be arranged in a circular pattern with diameters at 6, 7 and 8 millimeters.

The markers 16 may be attached to an actuator 20. The actuator 20 can be depressed to move the markers 16 into contact with the cornea. The markers 16 are typically dipped in a marking ink so that contact with the cornea leaves an ink mark. It is preferably to use an ink color that is dissimilar from the color of the cornea so that the surgeon can readily view the markings. Alternatively, the markers 16 are tipped and apply just enough pressure onto the corneal tissue to leave temporary indents that serve as markings. This embodiment can be used with or without marking ink.

The corneal marker 10 may include a spring 22 that moves the markers 16 back to the original position when the surgeon releases the actuator 20. The spring 22 also applies a marking force through the markers 16 that is consistent for each use of the marker 10. The marker 10 may include a bushing 24 that mechanically couples the actuator 20 to the frame 12. The bushing 24 may have a centering aperture 26 that is used to align the marker 10 with the center of the cornea. By way of example, the centering aperture 26 can be aligned with a light ring that is projected onto the cornea. The frame 12 and actuator 20 can be constructed from an optically transparent material such as a clear plastic so that the surgeon can see the cornea through the marker 10.

The frame 12 may have a pair of O-ring seals 28 on the bottom surface 14. The space between the O-rings 28 may be in fluid communication with a vacuum port 30. The vacuum port 30 may be coupled to a source of vacuum (not shown). The vacuum source may create a vacuum pressure between the bottom surface 14 and the cornea to maintain the position of the marker 10. Although a vacuum port is shown, it is to be understood that other means for maintaining the position of the marker 10, such as suction cups or protrusions, may be employed.

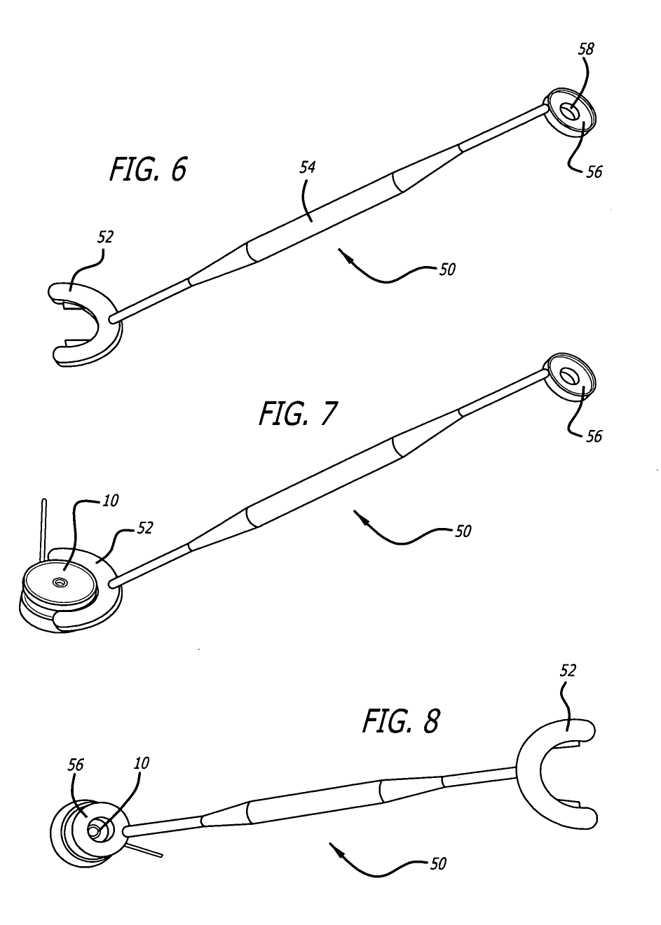

FIG. 6 shows a handle 50 that can be used by the surgeon to align the corneal marker with the cornea and then actuate the marker. The handle 50 may include a C-shaped first end 52 at one end of a handle shaft 54, and an annular shaped second end 56 at the other end of the shaft 54. The second end 56 may have an opening 58 that provides visual access during use of the handle 50.

In operation, a surgeon will initially dip the markers 16 into a reservoir of marking ink. As shown in FIG. 7, the surgeon then centers the marker 10 onto the cornea with the first end 52 of the handle 50. A vacuum can be created to maintain the position of the marker 10.

As shown in FIG. 8, the second end of the handle 50 can be used to depress the actuator 20. Depressing the actuator 20 pushes the markers 16 into the cornea. The vacuum is released and the marker 10 is then removed from the cornea. The marker 10 is able to create markings in multiple patterns with a single push of the actuator 20, thereby reducing the time required to mark the cornea.

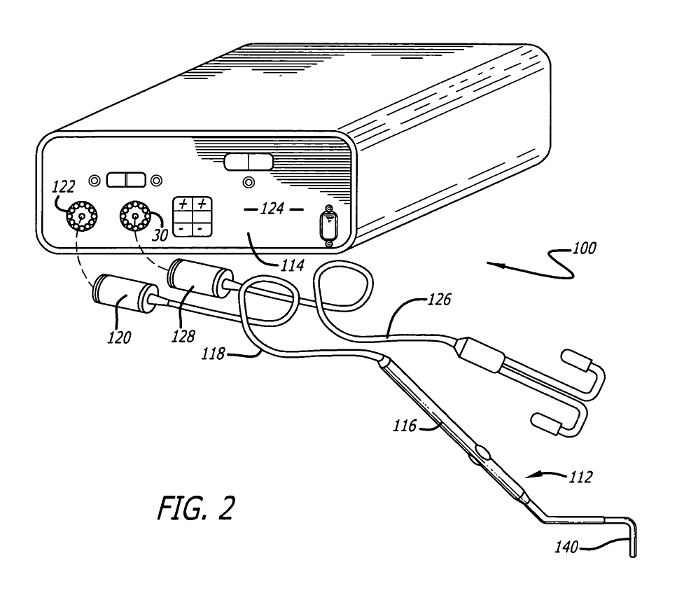

FIG. 2 shows a thermokeratoplasty electrode system 100 that can be used to create denatured areas in a cornea. Areas that are marked with the corneal marker 10 are shown in FIG. 1. The system 100 includes an electrode probe 112 coupled to a console 114. The console 114 contains a power supply that can deliver electrical power to the probe 112. The probe 112 has a hand piece 116 and wires 118 that couple the probe electrode to a connector 120 that plugs into a mating receptacle 122 located on the front panel 124 of the console 114. The hand piece 116 may be constructed from a non-conductive material.

The system 100 also includes a return element 126 that is in contact with the patient to provide a return path for the electrical current provided by the console 114 to the probe 112. The return element 126 has a connector 128 that plugs into a mating receptacle 130 located on the front panel 124 of the console 114. By way of example, the ground element may be a lid speculum that is used to maintain the patient's eyelids in an open position while providing a return path for the electrical current.

The console 114 provides a predetermined amount of energy, through a controlled application of power for a predetermined time duration. The console 114 may have manual controls that allow the user to select treatment parameters such as the power and time duration. The console 114 can also be constructed to provide an automated operation. The console 114 may have monitors and feedback systems for measuring physiologic tissue parameters such as tissue impedance, tissue temperature and other parameters, and adjust the output power of the radio frequency amplifier to accomplish the desired results.

In one embodiment, the console provides voltage limiting to prevent arcing. To protect the patient from over-voltage or overpower, the console 114 may have an upper voltage limit and/or upper power limit which terminates power to the probe when the output voltage or power of the unit exceeds a predetermined value.

The console 114 may also contain monitor and alarm circuits which monitors physiologic tissue parameters such as the resistance or impedance of the load and provides adjustments and/or an alarm when the resistance/impedance value exceeds and/or falls below predefined limits. The adjustment feature may change the voltage, current, and/or power delivered by the console such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature monitor. The front panel 124 of the console 114 typically contains meters and displays that provide an indication of the power, frequency, etc., of the power delivered to the probe.

The console 114 may deliver a radiofrequency (RF) power output in a frequency range of 100 KHz-5 MHz. In the preferred embodiment, power is provided to the probe at a frequency in the range of 350 KHz. The console 114 is designed so that the power supplied to the probe 112 does not exceed a certain upper limit of up to several watts. Preferably the console is set to have an upper power limit of 1.2 watts (W). The time duration of each application of power to a particular corneal location can be up to several seconds but is typically set between 0.1-1.0 seconds. The unit 14 is preferably set to deliver approximately 0.6 W of power for 0.6 seconds.

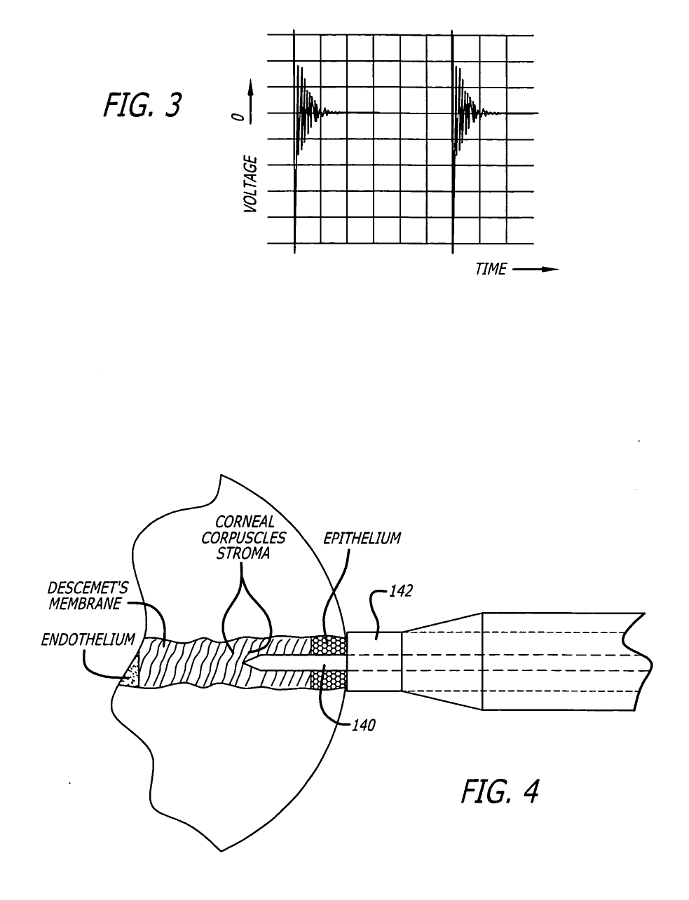

FIG. 3 shows a typical voltage waveform that is delivered by the probe 112 to the cornea. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

As shown in FIG. 4, during a procedure, an electrode tip 140 of the handpiece is inserted into a cornea. The length of the tip 140 is typically 300-600 microns, preferably 400 microns, so that the electrode enters the stroma layer of the cornea. The electrode may have a stop 142 that limits the penetration of the tip 140. The tip diameter is small to minimize the invasion of the eye.

The probe 112 provides a current to the cornea through the tip 140. The current denatures the collagen tissue of the stroma. Because the particular tip 140 is inserted into the stroma it has been found that a power no greater than 1.2 watts for a time duration no greater than 1.0 seconds will adequately denature the corneal tissue to provide optical correction of the eye. However, other power and time limits, in the range of several watts and seconds, respectively, can be used to effectively denature the corneal tissue. Inserting the tip 140 into the cornea provides improved repeatability over probes placed into contact with the surface of the cornea, by reducing the variances in the electrical characteristics of the epithelium and the outer surface of the cornea.

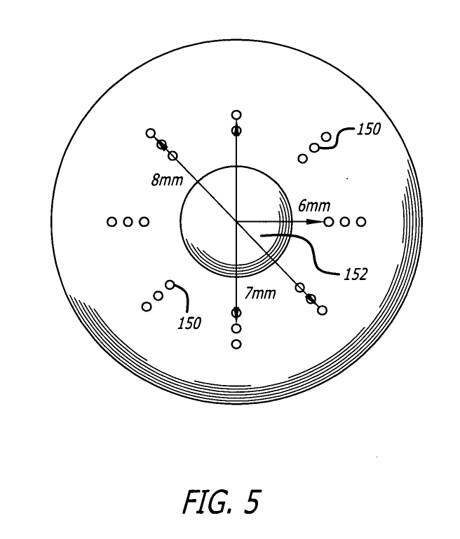

FIG. 5 shows a pattern of denatured areas 150 that have been found to correct hyperopic or presbyopic conditions. The denatured areas are marked by the corneal marker 10 shown in FIG. 1. A circle of 8, 16, or 24 denatured areas 50 are created about the center of the cornea, outside the visual axis portion 152 of the eye. The visual axis has a nominal diameter of approximately 5 millimeters. It has been found that 116 denatured areas provide the most corneal shrinkage and less post-op astigmatism effects from the procedure. The circle of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping. The assignee of the present application provides instructional services to educate those performing such procedures under the service marks CONDUCTIVE KERATOPLASTY and CK.

The exact diameter of the pattern may vary from patient to patient, it being understood that the denatured spots should preferably be formed in the non-visionary portion 152 of the eye. Although a circular pattern is shown, it is to be understood that the denatured areas may be located in any location and in any pattern. In addition to correcting for hyperopia and presbyopia, the present invention may be used to correct astigmatic conditions. For correcting astigmatic conditions, the denatured areas are typically created at the end of the astigmatic flat axis. The present invention may also be used to correct procedures that have overcorrected for a myopic condition.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC), microwave, ultrasonic and light can be transferred into the cornea. Non-thermal energy does not include the concept of heating a tip that had been inserted or is to be inserted into the cornea.

By way of example, the console can be modified to supply energy in the microwave frequency range or the ultrasonic frequency range. By way of example, the probe may have a helical microwave antenna with a diameter suitable for corneal delivery. The delivery of microwave energy could be achieved with or without corneal penetration, depending on the design of the antenna. The system may modulate the microwave energy in response to changes in the characteristic impedance.

For ultrasonic application, the probe would contain a transducer that is driven by the console and mechanically oscillates the tip. The system could monitor acoustic impedance and provide a corresponding feedback/regulation scheme For application of light the probe may contain some type of light guide that is inserted into the cornea and directs light into corneal tissue. The console would have means to generate light, preferably a coherent light source such as a laser, that can be delivered by the probe. The probe may include lens, waveguide and a photodiode that is used sense reflected light and monitor variations in the index of refraction, birefringence index of the cornea tissue as a way to monitor physiological changes and regulate power.

1. A corneal marker, comprising:
a frame;
a plurality of markers coupled to said frame; and, an actuator coupled to said frame and said markers.

2. The corneal marker of claim 1, further comprising a spring coupled to said frame and said actuator.

3. The corneal marker of claim 1, wherein said frame has a centering aperture.

4. The corneal marker of claim 1, wherein said frame includes a seal and a vacuum port.

5. The corneal marker of claim 1, wherein said markers are located at 6, 7 and 8 millimeters about a center of a cornea.

6. The corneal marker of claim 1, wherein said frame is optically transparent.

7. The corneal marker of claim 1, wherein said markers apply an ink that is dissimilar in appearance from a cornea marked by the corneal marker.

7a. The corneal marker of claim 1, wherein said markers produces marking indents into the corneal tissue.

8. The corneal marker of claim 1, further comprising a handle that can used to actuate said actuator.

9. The corneal marker of claim 1, further comprising a handle that can used to align said frame on the cornea.

10. A corneal marker for marking a cornea, comprising:
a frame;
a plurality of marking markers coupled to said frame; and,
means for moving said markers into contact with the cornea.

11. The corneal marker of claim 10, wherein said means includes an actuator and a spring that are coupled to said frame.

12. The corneal marker of claim 10, further comprising centering means for centering said frame onto the cornea.

13. The corneal marker of claim 10, further comprising fixation means for maintaining a position of said frame.

14. The corneal marker of claim 10, wherein said markers are located at 6, 7 and 8 millimeters about a center of a cornea.

15. The corneal marker of claim 10, wherein said frame is optically transparent.

16. The corneal marker of claim 10, wherein said markers apply an ink that is dissimilar in appearance from a cornea marked by the corneal marker.

16a. The corneal marker of claim 10, wherein said markers produces marking indents into the corneal tissue.

17. The corneal marker of claim 11, further comprising a handle that can used to actuate said actuator.

18. The corneal marker of claim 10, further comprising a handle that can used to align said frame on the cornea.

19. A method for marking a cornea, comprising:
centering a frame onto a cornea, the frame holding a plurality of markers; and,
moving the markers into contact with the cornea.

20. The method of claim 19, wherein the markers are pushed into the cornea by actuating an actuator.

21. The method of claim 19, wherein the frame is centered by aligning a centering aperture of the frame with a ring light projected onto the cornea.

22. The method of claim 19, further comprising maintaining a position of the frame with a vacuum pressure between the frame and the cornea.

23. The method of claim 19, wherein the markers apply an ink that is dissimilar in appearance from the cornea.

24. The method of claim 19, wherein the markers leave a plurality of marks on the cornea at 6, 7 and 8 millimeters about a center of the cornea.

25. The method of claim 19, wherein the actuator is actuated with a handle.

26. The method of claim 19, wherein the frame is centered with a handle.

27. A handle that is used with a corneal marker, comprising:
a shaft that has a C-shaped first end and an annular shaped second end.

28. The handle of claim 27, wherein said annular shaped second end has an opening.
